(19) United States
(12) Patent Application Publication
(10) Pub. No.: US 2008/0139654 A1
(43) Pub. Date: Jun. 12, 2008

(54) ACETAMINOPHEN COMPOSITIONS HAVING MINIMIZED SIDE EFFECTS INCLUDING REDUCED HEPATOTOXICITY

(76) Inventor: Eric Mott Soderling, Discovery Bay, CA (US)

Correspondence Address: DANIEL B. SCHEIN, PH.D., ESQ., INC., P. O. BOX 68128, Virginia Beach, VA, 23471, US

(21) Appl. No.: 11/952,556

(22) Filed: Dec. 7, 2007

Related U.S. Application Data
(60) Provisional application No. 60/873,743, filed on Dec. 9, 2006.

(51) Int. Cl. A61K 31/198; A61K 31/16; A61K 9/48

(52) U.S. Cl. 514/562; 514/629; 424/451

(57) ABSTRACT

Solid tablets or gel capsules comprising acetaminophen and an agent that promotes glutathione production that mitigates adverse hepatic effects of acetaminophen. The glutathione production promoter is preferably n-acetylcysteine or other mercapto-2-amino alkyl carboxylic acid having glutathione production promoting properties. A preferred composition comprises acetaminophen (200 mg to 750 mg) and N-acetylcysteine (200 mg to 600 mg). Alternatively and/or in addition to N-acetylcysteine the composition can contain at least one of methionine and cysteine. Preferred compositions can contain at least one of an opiate (or synthetic equivalent), an antihistamine, an antiemetic, and a sedative. Physical encapsulation of the ingredients optionally with or in place of a fragrance is used to make the composition more acceptable to patients by mitigating noxious properties of the glutathione production promoter. Preferably, the compositions are prepared for patient self administration in tablet or gel capsule form wherein the patients can take the medication without the need for close oversight of a medical caregiver.

### PRIORITY

The present application claims priority of U.S. provisional patent application Ser. No. 60/873,743, filed Dec. 9, 2006.

### FIELD OF THE INVENTION

The present inventions relate to acetaminophen compositions and methods for administering same to treat pain and other conditions for which acetaminophen administration is desired (e.g., antipyretic treatment) while minimizing multiple side effects including liver toxicity (hepatotoxicity) associated with acetaminophen. In a preferred embodiment, the present inventions are directed to solid tablets or gel capsules comprising acetaminophen and an agent that promotes glutathione production. The glutathione production promoter is preferably n-acetylcysteine or other mercapto-2-amino alkyl carboxylic acid having glutathione production promoting properties shown to mitigate acetaminophen-induced hepatotoxicity.

In a preferred embodiment, compositions of the present invention also include an antiemetic and/or an antihistamine. The embodiments with an antiemetic and/or antihistamine will mitigate acetaminophen and/or opiate related nausea and vomiting (several formulations currently exist combining acetaminophen with opiates). The embodiments with antihistamines will also reduce itching and other histamine-based side effects known to be associated with acetaminophen and opiates. Antihistamines are also known to have an analgesic property improving the total analgesic effect over acetaminophen alone. Some antihistamines have sedating properties inducing sleep which can be useful in some circumstances in patients experiencing pain. Preferably, the compositions are prepared for patient self-administration in tablet or gel capsule form wherein the patients can take the medication without the need for close oversight of a medical caregiver. These embodiments will contain fragrances and/or physical encapsulation that will mitigate the smell and taste of the preparations improving patient compliance, as the glutathione promoters have a noxious smell and taste along with other side effects, which has thus far limited their use.

### BACKGROUND OF THE INVENTION

N-acetyl-p-aminophenol, also referred to generically as acetaminophen and sold under the trademark TYLENOL inter alia is one of the most common pain relievers and antipyretics sold and used in the United States and around the world. Prolonged use or ingestion of acutely elevated levels of N-acetyl-p-aminophenol can result in liver damage. As used herein acetaminophen includes pharmaceutically equivalent analogs of acetaminophen. Without being limited to any particular theory of biological activity, it is believed that the hepatotoxic effects of acetaminophen are related to intracellular depletion of glutathione reserves. This allows for the accumulation of the toxic metabolite N-acetyl-benzoquinoneimine. As this substance increases intracellularly it covalently binds to the lipid bilayer of hepatocytes resulting in hepatic centrilobular necrosis. Left untreated this mechanism leads to substantial morbidity and mortality. Regardless of the mechanism of action, acetaminophen is considered to be the cause of severe liver damage and death in many patients.

N-acetylcysteine (hereinafter also referred to as “NAC”) is the only pharmacological agent currently accepted for use in the treatment of N-acetyl-p-aminophenol intoxication. See Goodman and Gilman's The Pharmacological Basis of Therapeutics, 7th edition, 1985, MacMillan, inter alia.

N-acetylcysteine, a mercapto-2-amino-alkyl carboxylic acid derivative, is the rate-limiting substrate in the intracellular formation of glutathione. Glutathione is thought to be impermeable to hepatocytes and is not an effective antidote to N-acetyl-p-aminophenol. Numerous studies have demonstrated that N-acetylcysteine is an effective antidote to N-acetyl-p-aminophenol-related hepatotoxicity.

N-acetyl-p-aminophenol is available for use as an elixir or in solid form. N-acetyl-p-aminophenol is also commonly combined with opiates to make a more potent analgesic (e.g., a composition of hydrocodone and acetaminophen are available as Vicodin®). Due to the addictive properties of opiates, medications containing acetaminophen and opiates are commonly abused. A well-documented effect of opiates is the rapid tolerance developed after only a short period of use (e.g., less than 3 days depending on the dosage). In order to maintain the same analgesic effect, patients often escalate their doses, generally doing so despite being warned against this by their physician. This can lead to inadvertent overdose of N-acetyl-p-aminophenol, which can lead to severe liver damage and even death. Consuming alcohol while taking acetaminophen has been linked to severe liver damage. Some patients administered “safe” dosages of acetaminophen still develop severe hepatotoxicity. For example, an article in the Journal of Internal Medicine (2003): 253; pp. 240-243, reports a case of a 53 year old female who developed severe hepatotoxicity after receiving Tylenol at the recommended dose of 4 grams/day. There are also combinations of acetaminophen with other pain relievers, for example Tramadol.

N-acetyl-p-aminophenol overdose is initially clinically unremarkable and the patient is completely asymptomatic in their early presentation in many cases. This often delays the patient from identifying the overdose and seeking timely treatment for the toxic effects of acetaminophen. In many instances, the damage to the liver is done several days before a patient seeks treatment or even longer.

Despite the availability of N-acetylcysteine for treating the N-acetyl-p-aminophenol-induced hepatotoxicity, N-acetyl-p-aminophenol still remains the most common pharmacological agent resulting in overdose deaths and the second most common cause for hepatic failure in pediatric patients. This can be attributed in some instances to emesis and nausea in patients with hepatotoxicity that is exacerbated by nausea associated with the noxious smell and taste of N-acetylcysteine (NAC). A patient suffering from the effects of excessive alcohol and/or acetaminophen consumption will find it difficult to ingest NAC due to its noxious physical properties, with the noxious taste and smell being obvious and nauseating to many patients. Further, NAC administered via IV has been associated with anaphylactic shock.

Acetaminophen at recommended doses is usually a very safe and useful drug that can effectively and safely reduce fever and pain for most patients. It is also widely available and inexpensive. Therefore, despite the large numbers of deaths and cases of liver damage requiring transplant and/or treatment associated with the use of acetaminophen, doctors continue and will likely continue to prescribe medicines containing acetaminophen and many individuals will continue to buy acetaminophen over the counter without a prescription and drink alcohol concomitantly and/or take more acetaminophen than is safe.

As noted above, NAC is known to treat the harmful effects on the liver of acetaminophen, yet its use has been limited to treating patients after liver damage is found. Further, the use of NAC is discouraged in post-operative patients since they may vomit when administered NAC causing rupture of stitches. In all patients, vomiting due to NAC administration can lead to loss of other needed medications and nourishment and physical injury. Since IV use of NAC can also cause anaphylactic shock, NAC use has been limited to treating liver damage after it has been detected. Due to NAC's emetic and anaphylactic shock potential, its use has been limited to treating liver disease caused by acetaminophen toxicity and as a mucolytic for pulmonary congestion.

The physical properties of these preparations with the introduction of fragrances and/or encapsulation, by various means, will serve to mitigate the noxious taste and smell of NAC, and other glutathione promoters, thereby also reducing the nausea and vomiting.

The inclusion of an antiemetic, or an antihistamine with antiemetic properties, will mitigate the nausea and vomiting associated with N-acetyl-p-aminophenol, NAC, and opiates.

The embodiments with antihistamines will also reduce itching and other histamine-based side effects known to be associated with acetaminophen and opiates. Antihistamines are also known to have an analgesic property improving the total analgesic effect over acetaminophen alone. Some antihistamines have sedating properties inducing sleep which can be useful in some circumstances in patients experiencing pain.

There is a need to minimize and preferably eliminate the risk of liver damage from ingestion of acetaminophen, whether due solely to acetaminophen ingestion and/or concomitant use of alcohol.

There have been numerous research studies directed to how to address acetaminophen-induced hepatoxicity, sometimes providing contradictory teachings. For example, the article “Controversies in management: Should methionine be added to every paracetamol tablet?,” BMJ 1997; 315:301-303 (2 August), teaches away from adding methionine to acetaminophen compositions for fear of toxicity, inter alia; the article indicates that, while whether long term increased intake of methionine could promote carcinogenesis has not been evaluated, methionine is metabolised to homocysteine and vice versa in a methylation cycle, and raised plasma homocysteine concentrations have been associated with peripheral vascular disease, ischaemic heart disease, and stroke . . . ; coagulation abnormalities; vascular injury; endothelial dysfunction; and vascular smooth muscle proliferation. Oral administration of methionine (200 mg/kg/day) to rats for 14 days increased the concentrations of homocysteine in serum and produced angiotoxic effects similar to atherosclerosis.

“MUCOMYST®, Acetylcysteine. Mucolytic—Antidote for Acetaminophen Poisoning,” Roberts, http://www.rxmed.com/b.main/b2.pharmaceutical/b2.1.monographs/CPS-%20Monographs/CPS-%20 (General%20Monographs-%20M)/MUCOMYST.html, indicates that large overdose of acetaminophen (150 mg.kg or greater) saturates the glucuronide and sulfate conjugation pathways, leading to increased formation of reactive metabolite that depletes glutathione, leading to hepatic cellular necrosis [liver cell death]. Acetylcysteine is believed to protect the liver by maintaining or restoring glutathione inter alia Occasionally severe and persistent vomiting occurs as a symptom of acute acetaminophen overdose. Treatment of oral acetylcysteine may aggravate the vomiting. (Emphasis added). However, as noted before, exogenous glutathione administration is not known to be an effective treatment for acetaminophen induced hepatotoxicity.

U.S. Pat. No. 5,474,757, issued Dec. 12, 1995, teaches prevention of acetaminophen overdose by use of organosulfur compounds, specifically, diallyl sufide and diallyl sulfone, to prevent formation of hepatotoxic metabolites from acetaminophen. Yet, despite stating that the composition can prevent formation of hepatotoxic metabolites from acetaminophen based on animal studies, it is suggested that the compositions could further include N-acetylcysteine. Methionine is also taught as an alternative to N-acetylcysteine despite the teachings of adverse side effects mentioned above, and the noxious aspects of the compounds were not addressed.

However, dially sulfide (or “DAS”) is well known to have a powerful noxious odor. For example, in “Diallyl Sulfide (Funny how tasty compounds come in bottles labeled “STENCH” when it's the pure chemical),” Molecule of the Day, Sep. 13, 2006, it is noted that diallyl “sulfide is a chemical that you would like to avoid neat [e.g., in concentrated form] However, in small quantities, it's responsible for a fair bit of the aroma of garlic.” N-acetylcysteine also has a “stench” that can cause vomiting and/or an allergic reaction. Therefore, human patients, unlike laboratory rats, are unlikely to ingest a noxious composition when more palatable alternatives are available. A concept ignored by the prior art is that compositions containing an extra ingredient primarily designed to avoid the effects of overdosing and/or concomitant alcohol consumption are unlikely to appeal to most patients. Even if the cost is low of combining diallyl sulfide and/or N-acetylcysteine with standard acetaminophen compositions, patients and medical insurers will tend to use the less expensive composition that does not have a noxious odor, cause nausea, and/or have other well know side effects. This situation is particularly grave for patients taking acetaminophen combined with opioids or analogous compounds. The fear of admitting to a craving for larger dosages of acetaminophen compositions containing opioids, or fear that a doctor may not prescribe a preferred pain medication for patients already taking large dosages, is likely to lead many patients not to tell their doctor about how much they take or about concomitant alcohol consumption.

Thus, there is a long felt, but unmet need, for acetaminophen compositions that will not cause hepatotoxicity while avoiding or minimizing noxious odors, and that automatically address psychological factors that cause acetaminophen to be used in an unsafe manner. There is also a need for physicians and pharmaceutical companies to recognize and address the factors that lead patients to unnecessarily suffer acetaminophen induced hepatotoxicity.

Further background information on acetaminophen poisoning and acetylcysteine can be found in “Treatment of Acetaminophen Poisoning,” Kaplowitz, Neal, M.D., Liver Newsletter, University of Southern California Department of Surciery, www.surgery.usc.edu/divisions/hep/livernewsletter-treatmentofacetaminophenpoisoning.html; “Acetylcysteine” from Wikipedia, http://en.wikipedia.org/wiki/Acetylcysteine; and “Toxicity, Acetaminophen,” Farrell et al., Emedicine, http://www.emedicine.com/emerg/topic819.htm.

### SUMMARY OF THE INVENTION

An embodiment of the present inventions is a composition of acetaminophen combined with an agent that promotes glutathione production in a form that patients would be willing to take. The addition of fragrances and/or physical encapsulation by various means will mitigate the noxious smell and taste of the glutathione production promoter to the extent that patients are willing to voluntarily take the medication. Currently, patients threatened with the possibility of death from acetaminophen-induced-hepatotoxicity have refused to take NAC due to its noxious physical properties. The prior art has not addressed why a patient would take a potentially dangerous and noxious medication for simple analgesia when many have refused to take it to save their lives and/or avoid serious adverse heath effects.

The dosage of the glutathione production promoter is low enough in preferred compositions of the present inventions that more than about 100 to 500 times the safe dosage of acetaminophen must be taken via ingestion of the composition to approach the toxic dosage of the glutathione production promoter, provided a sufficient dosage of the glutathione production promoter is administered. In an embodiment, the composition of the present invention comprises acetaminophen combined with at least one mercapto-2-amino alkyl carboxylic acid having glutathione production promoting properties, wherein, a mammal will have substantially none or reduced hepatotoxic effects after consuming a sufficient amount of the composition to ingest an amount of acetaminophen sufficient to cause hepatotoxic effects in the absence of the mercapto-2-amino alkyl carboxylic acid. In an embodiment, the preferred glutathione producing agent is N-acetylcysteine (“NAC”), and is compounded in pill or capsule form with acetaminophen. In accordance with an aspect of the present inventions NAC is compounded with standard dosages of acetaminophen that are currently available (e.g., 325 or 650 mg acetaminophen), wherein the composition of the present invention comprises acetaminophen and NAC. It has been surprisingly discovered that small doses of a glutathione production promoting agent, such as NAC, can be combined in tablet or capsule form with acetaminophen with minimal or no nausea occurring during oral administration. In instances where patients increase the dosage of the composition due to inclusion of a narcotic in the formulation, the dosage of NAC is automatically increased, thus concomitantly minimizing or avoiding hepatotoxicity. It has also been surprisingly discovered that concerns about hepatotoxic effects that have caused physicians to recommend lower doses of acetaminophen than needed to achieve adequate analgesic or antipyretic effects that are overcome by the present invention. The compositions of the present inventions can be administered so that more than about 4 grams of acetaminophen can be delivered per day to a healthy adult mammal (e.g., human) weighing at least 100 pounds without irreparable hepatic toxicity effects. In an alternative embodiment, all acetaminophen tablets and capsules containing an opioid or other substance that is subject to abuse must contain a sufficient amount of N-acetylcysteine to prevent hepatoxicity, preferrably at dosages of 4 grams of acetaminophen per day. Further, prescribing literature will be modified to reflect the assumption that patients will exceed recommended dosages of acetaminophen when combined with an opioid or other addictive agent. Under such an assumption, it becomes appropriate to require the addition of a prophylactic agent to prevent hepatotoxicity to acetaminophen compositions, and that medical insurance cover the additional cost. Patient compliance is enhanced by reducing the noxious odor and taste of the prophylactic agent(s). Further, the additional costs of the prophylactic agent(s) may be more than offset by the cost savings in reducing the number of patients requiring treatment for hepatotoxicity.

In other embodiments, acetaminophen is combined with at least one compound from the group consisting of NAC, methionine, and cysteine. In additional embodiments of the present invention, in addition to acetaminophen and NAC, the composition comprises at least one active agent selected from the group consisting of a narcotic drug (e.g., codeine, hydrocodone), an anti-emetic drug, an antihistamine drug, and an anti-inflammatory drug. Antihistamines also act as anti-emetics, anti-pruritics, soporifics, and mild analgesics. These mitigate the known side effects of acetaminophen, and/or glutathione promoters, and/or opiates. Mitigation of these side effects will also increase patient compliance. Antihistamines and anti-inflammatories analgesic effects will make for a more effective analgesic.

### FURTHER DESCRIPTION OF THE INVENTIONS

In producing embodiments of the present invention, the forgoing summary and other materials are supplemented and expanded by the following further description of the inventions, it being understood that the summary is not repeated hereafter in order to facilitate economy of description. Embodiments of the present inventions can include various combinations of various classes of drugs may, for example: acetaminophen can be combined with NAC and optionally DAS, and also be combined with stimulants such as caffeine, Antihistamines (H1 and H2 blockers), NSAID's, proton-pump inhibitors, laxatives, antiemetics, opiates and other analgesics, anxiolytics, muscle relaxants. Exemplary members of each the forgoing classes of drugs include but are not limited to:

1) for antihepatoxic effects, drugs with a sulfhydryl moiety, such as NAC, Methionine, L-cysteine;
2) stimulants and drugs with CNS stimulating effects, such as caffeine, theophylline, dextroamphetamine, amphetamine, methamphetamine, atomoxetine, dexmethylphenidate, methylphenidate, modafinil, phentermine, and sibutramine;
3) antihistamines that have an H1-blockers effect, such as desloratidine, fexofenadine, loratidine, azatidine, cetirizine, chlorfeniramine, clemastine, cyproheptadine, dexchlorpheniramine, diphenhydramine, hydroxyzine, promethazine;
4) antihistamines that have an H2-blocker effect and/or antacid effect, such as: cimetidine, famotidine, nizatidine, ranitidine;
5) antinflamtory drugs such as nonsteroidal antiflamatory drugs, aspirin, diflunisal, salsalate, trilisate, flurbiprofen, ibuprofen, ketoprofen, naproxen, oxaprozin, diclofenac, etodolac, indomethacin, ketorolac, nabumetone, sulindac, tolmetin, meclofenamate, meloxicam, piroxicam, celecoxib;.
6) proton-pump inhibitors and/or antacids, such as esomeprazole, lansoprazole, omeprazole, pantoprazole, rabeprazole, or any other drug with proton-pump inhibitor or antacid effects;
7) antiemetics, such as apreitant, doxylamine, pyridoxine, dimenhydrinate, dronabinol, drpperidol, meclizine, metoclopramide, phosphorated carbohydrates, prochlorperazine, promethazine, scopolamine, thiethylperazine, and trimethobenzamide;

### 8) Laxatives, such as bisacodyl, cascara, docusate calcium, docusate sodium, magnesium citrate, magnesium hydroxide, sennosides, polyethylene glycol, senna, and sorbitol;

### 9) Drugs with 5-HT3-blocker effects, such as antiemetics that are 5-HT3-blockers, and other 5-HT3-blockers, such as dolasetron, granisetron, ondansetron, and palonosetron.

10) Opiates and other analgesics, such as buprenophine, butorphanol, nalbuphine, pentazocine, codeine, fentanyl, hydromorphone, levorphanol, meperidine, methadone, morphine, oxycodone, oxymorphone, propoxyphene, hydrocodone, dihydrcodeine, nalmefene, naloxone, tramadol, and ziconotide;
11) Anxiolytics, such as bromazepam, chlordiazepoxide, clonazepam, chlorazepate, diazepam, flurazepam, estazolam, lorazepam, temazepam, alprazolam, oxazepam, triazolam, buspironechloral hydrate, diphenhydramine, eszoclipone, ramelteon, zaleplon, zolpidem, and zipiclone; and

### 12) Muscle relaxants, such as baclofen, carisoprodol, chlorzoxazone, cyclobenzaprine, dantrolene, diazepam, metaxalone, methocarbamol, orphenadrine, and tizanidine.

For the purposes of describing the present inventions, drugs may have more than one of the 12 effects listed above and/or belong to more than one class of drugs listed above, regardless of how these drugs might otherwise be classified in the art and/or any particular mechanism of action.

Preferred embodiments of the present invention comprise acetaminophen, NAC and at least one of an antihistamine and an antiemetic. Preferrably, the compositions, with or without the addition of a fragrance of the present invention are in solid pill or capsule form, wherein the noxious odor and taste of NAC can be minimized and nausea offset by the presence of an antiemetic. Coatings designed to break down after swallowing may be included in some formulations.

The present invention also provides for an adjunctive composition to prevent hepatotoxicity, and other related side effects. For example, a patient taking a liquid elixir of acetaminophen or a liquid medicine such as cold medications containing acetaminophen would not want to include in medication the liquid form of NAC due to the noxious odor and possible emesis that can create related health complications. Patients may also be prescribed or purchase acetaminophen containing pills that contain nothing to offset possible hepatotoxic effects. In an alternative embodiment, the present invention provides a method for preventing hepatoxicity in patients taking acetaminophen via providing sufficient dosages of NAC in solid pill or capsule form at or near the time of each dosage of acetaminophen given. The tablet is preferably formulated to reduce the odor of NAC, such as by coating, use of masking agents, or compounding with compounds that offset or absorb the NAC odor. Preferably, the solid form of NAC is combined with an antiemetic and/or an antihistamine.

Examples of testing and use of the present inventions are provided to expedite making and using the compositions of the present inventions.

### EXAMPLE 1

A 37 year-old male construction worker* fell from a height of about 8 feet, severely breaking his right ankle and wrist. (*to promote privacy and simplify description of the invention, the patient and symptoms described here are a composite of data from typical patients). Orthopedic surgery repaired the injury, followed by prolonged and painful rehabilitation. A prescription for 60 Vicodin tablets (a composition comprising acetaminophen and hydrocodone) is given routinely during such post-surgical recovery periods. Typcially, the first day 4 to 8 vicodin tablets manage pain (often in combination with sedatives and the aftereffects of surgical anesthetics).

On the second day, at least 8 Vicodin® tablets are generally required to get the same analgesic effect (Vicodin is a well known trademark, which will also be referred to herein by “vicodin”). Subsequently, it is common that more than 10 vicodin tablets per day are taken, contrary to the doctor's instructions, yet less pain relief is noted with this higher dosage. At this rate, the patient must seek a refill in only a week; often doctors refilling such prescriptions give a stern advisory about taking too many pills and the dangers of Tylenol/acetaminophen. Subsequently, the patient sought prescriptions from other physicians and took 30 Vicodin tablets each day. After two days, the patient visited the emergency room and received a shot of morphine and another Vicodin prescription. As is common, no disclosure was made to any physician that the patient also consumes about 8 beers each day. About one day later, the patient was vomiting and was taken to the emergency room. A routine set of lab tests revealed significant elevated liver enzymes. This prompted closer questioning by the emergency room doctor who then started the patient on Mucomyst (n-acetylcysteine) and ordered acetaminophen levels. Despite the treatment with Mucomyst the patient's liver enzymes remained elevated two weeks later and he was referred to a Gastroenterologist. Chronic alcohol abuse and acute Tylenol overdose resulted in the destruction of over 90% of his liver, thus requiring a liver transplant within the year, but alcoholism precluded placement on a transplant list. Death attributed to acetaminophen and alcohol occurred about 13 months later.

### EXAMPLE 2

A 26 year-old female college student* felt run down and suffered several colds during the prior winter (*to promote privacy, the patient and symptoms described here are a composite of data from typical patients). She found that Nyquil cough syrup at night helped her cough, and also let her sleep better. She didn't read the instructions carefully and would just take “a gulp” 3 times a night. Her cough didn't improve so she started taking Dayquil cough syrup during the day too. A week later she started to vomit and thought she had caught the flu. She saw her physician who with a careful history diagnosed Tylenol poisoning and admitted her to the hospital. She was unable to tolerate the taste and smell of Mucomyst, so a nasogastric tube was inserted for its administration. She was discharged 2 days later and made a full recovery.

### Effect of Prophylactic Administration of N-Acetylcysteine on Acetaminophen Toxicity in the Rat

### Phase I Trial

The effect of prophylactic administration of n-acetylcysteine (NAC) on acetaminophen hepatotoxicity can be determined in the rat. In a double-blind placebo study, four groups of rats are given one of the following oral dosings: “NAC-free” (NF), low NAC (LN), medium NAC (MN), or high NAC(HN). Then rats in all groups are administered a toxic dose of acetaminophen (1 g/kg body weight) orally. After 24 hours, animal are euthanized and assessed for hepatotoxicity by the elevation of serum transaminases and by a necrotic score based on histological examination. With animal studies establishing a hepatoprotective effect of prophylactic NAC, a Phase II trial is conducted as described below.

### Determining Optimum Dosing in the Prophylactic Administration of N-Acetylcysteine on Acetaminophen Toxicity in the Rat

### Phase II Trial

It is desirable to determine the optimum dosing of prophylactic NAC administration required to protect against acetaminophen hepatotoxicty. NAC has significant side effects, including but not limited to, vomiting, nausea, and flushing, and anaphylactic shock. Some of these undesired effects are shared with acetaminophen toxicity. Thus, it is advantageous to establish the minimum possible dosing to provide a hepatoprotective effect against acetaminophen, while minimizing serious side effects. In the following protocol, all rats in the study are given the LD50 dose of acetaminophen (determined by the dosage of acetaminophen that causes 50% of the test subjects to expire after a predetermined period of time). While administering a consistent LD50 dosage of acetaminophen (0.5 g/kg of Body weight), varying doses of NAC are administered just prior to administration of the LD50 doses of acetaminophen. The study is conducted in double-blind fashion; four groups of rats are given one of the following oral dosings: “NAC-free” (NF or placebo), low NAC (LN), medium NAC (MN), or high NAC(HN). Then rats in all groups are administered a LD50 dose of acetaminophen. All animals are euthanized 3 weeks later and hepatotoxicity assessed by the elevation of serum transaminases and by a necrotic score based on histological examination. Mortality in all groups will also be followed as the added criterion.

Experimental data supports that concomitant administration of acetaminophen with an agent that has glutathione production promoting properties reduces or avoids hepatoxicity. Preferably, a mercapto-2-amino alkyl carboxylic acid having glutathione production promoting properties is coadministered in pill or capsule form with acetaminophen. In a preferred embodiment, n-acetylcysteine is combined with acetaminophen in formulations made in accordance with Table I below.

TABLE 1
EXEMPLARY ACETAMINOPHEN COMPOSITIONS
HAVING REDUCED HEPATOTOXICITY

| INGREDIENT | AMOUNT | EFFECT |
|---|---|---|
| Acetaminophen | 200 mg to 750 mg | Pain relief, antipyretic |
| N-acetylcysteine | 200 mg to 600 mg | Glutathione production |
| | | promoter; liver |
| | | protectant; mucolytic |

OPTIONAL INGREDIENTS

| | | |
|---|---|---|
| Dimenhydrinate* | 10 to 200 mg | Antiemetic |
| (Diphenhydramine, | | |
| Loratidine or Phenergan | | |
| can also be used with | | |
| dose adjusted | | |
| accordingly), | | |
| Hydrocodone** | 5 mg to 10 mg | Pain relief |
| (codeine, morphine, | | |
| propoxyphene can also | | |
| be used with dose | | |
| adjusted accordingly) | | |
| Manufacturer's choice | | Sedative* |
| Manufacturer's choice | | excipient |
| Manufacturer's choice | | Binder |
| Manufacturer's choice | | Flavoring |
| Manufacturer's choice | | buffer |

*antihistamines can be sedating (i.e., soporific) or non-sedating. A sedating antihistamine (e.g., Diphenhydramine) could be used in an alternative formulation for night use when sedation is desired.
**may substitute other synthetic or natural opiate receptor agonist. Gelatin, water and magnesium stearate can be used as in conventional tablet and capsule manufacture.

While N-acetylcysteine has adverse side effects at large dosages, a very large amount of an acetaminophen-containing composition of the present invention would have to be consumed to approach the toxic level of N-acetylcysteine; however, in order to consume such a large quantity of the present invention other ingredients would cause noticeable effects encouraging the user to stop taking more. For example, an agent could be added that is not noticeable at low doses but causes vomiting only at high dosages. Also, agents that offset the euphoria of addictive drugs may also be used to discourage abuse as are known to those of skill in the art.

In an embodiment, after determination of the hepatotoxic threshold of acetaminophen, a sufficient amount of n-acetylcysteine or other agent that counters the hepatotoxic effects of acetaminophen is added to that amount of acetaminophen, with the mix then divided into standard aliquots sufficient to meet the needs of a patient. In this way, hepatotoxicity risks associated with acetaminophen are minimized if not eliminated. With reduced fear of hepatotoxicity, doctors will feel more comfortable prescribing higher dosages of acetaminophen containing compositions to adequately treat fevers and pain, while patients should increase compliance with doctor's orders to take medication containing acetaminophen knowing that the well-publicized liver toxicity concerns are being addressed.

Further, since acetaminophen is an inexpensive drug, use of the compositions of the present inventions will reduce drug costs and also decrease costs associated with liver toxicity treatment (which can lead to expensive liver transplants), whereas the alternative may be in some cases death. Further, since n-acetylcysteine is a mucolytic, the compositions of the present inventions would make an ideal medication for pulmonary conditions in addition to its usefulness for treatment of fever and painful bodily injuries.

Thus, exemplary embodiments and uses of the present inventions have been described. Alternative embodiments, descriptions and terms are contemplated. While exemplary embodiments of the present invention have been set forth above, it is to be understood that the pioneer inventions disclosed herein may be made and used otherwise than as specifically described.

1. A composition for the treatment of pain or other aliment in a mammal that is subject to treatment with acetaminophen, comprising acetaminophen and an effective amount of a glutathione production promoter, wherein hepatotoxic effects of acetaminophen administration are reduced with respect to administration of an equal amount of acetaminophen without a glutathione production promoter, wherein said composition can be administered so that at least about 4 grams of acetaminophen can be administered per day to an adult mammal weighing at least 100 pounds without irreparable hepatic toxicity effects.

2. The composition of claim 1, wherein said glutathione production promoter comprises n-acetylcysteine.

3. A composition for the treatment of pain or other aliment that is subject to treatment with acetaminophen, comprising acetaminophen and n-acetylcysteine, wherein a patient taking an amount of said composition containing a dosage of acetaminophen known to cause hepatotoxicity will have no hepatotoxicity or insufficient hepatotoxicity to require additional treatment for hepatotoxicity.

4. A composition comprising acetaminophen and n-acetylcysteine, said composition formulated to decrease emesis associated with n-acetyltcysteine administration.

5. The composition of claim 4, wherein said composition comprises at least one of the group consisting of an antiemetic drug and ingredients to reduce the smell associated with n-acetylcysteine.

6. The composition of claim 4 formulated in a solid composition or capsule.

7. The composition of claim 4, further comprising an antihistamine.

8. The composition of claim 7, further comprising an antiemetic drug.

9. The composition of any of claims 4-8, further comprising a narcotic drug.

10. The composition of any of claims 4-8, further comprising at least one compound selected from the group consisting of stimulants, H1 blockers, H2 blockers, antinflamtories, proton-pump inhibitors, laxatives, 5-HT3-blockers, anxiolytics, and muscle relaxants.

11. A method for decreasing hepatotoxicty associated with administration of acetaminophen, comprising coadmistration of n-acetyl cysteine and acetaminophen, wherein said coadministration is simultaneous or n-acetylcysteine is administered in a sufficiently short amount of time before or after acetaminophen administration to decrease hepatotoxicity in comparison to administration of acetaminophen in the absence of n-acetylcysteine.

12. The method of claim 11, wherein acetaminophen is administered separately from n-acetylcysteine, and n-acetylcysteine is administered in a solid tablet or capsule that is formulated to decrease emesis associated with n-acetyltcysteine administration.

13. The method of claim 12, wherein acetaminophen is administered in liquid form.

14. A method for decreasing hepatoxicity associated with an extended course of administration of acetaminophen to a mammal, comprising the simultaneous, sequential or separate provision of doses of acetaminophen to a mammal, wherein the doses comprise at least about 4 grams per day of acetamimophen, and the period of said extended course exceeds at least one day.

15. The method of claim 14, wherein the period of said extended course is selected from the group consisting a period that exceeds one week, a period that exceeds one month, a period that exceeds one year, a period that is less than one week, a period that is less than one month but more than one week, a period that is less than one year but more than one month.

16. The composition of claim 9, further comprising at least one compound selected from the group consisting of stimulants, H1 blockers, H2 blockers, antinflamtories, proton-pump inhibitors, laxatives, 5-HT3-blockers, anxiolytics, and muscle relaxants.

17. The composition of claim 2, further comprising a fragrance.

18. The composition of claims 4-8, further comprising a fragrance.

19. The composition of claim 9, further comprising a fragrance, and at least one compound selected from the group consisting of stimulants, H1 blockers, H2 blockers, antinflamtories, proton-pump inhibitors, laxatives, 5-HT3-blockers, anxiolytics, and muscle relaxants.
